(19) United States
(12) Patent Application Publication
(10) Pub. No.: US 2015/0268158 A1
(43) Pub. Date: Sep. 24, 2015

(54) Gas Sensor and Method for Sensing Presence of Ethanol Vapor in a Cabin

(71) Applicant: Battelle Memorial Institute, Columbus, OH (US)

(72) Inventor: John S. Laudo, Hilliard, OH (US)

(73) Assignee: BATTELLE MEMORIAL INSTITUTE, Columbus, OH (US)

(21) Appl. No.: 14/661,494

(22) Filed: Mar. 18, 2015

Related U.S. Application Data
(60) Provisional application No. 61/968,537, filed on Mar. 21, 2014.

(51) Int. Cl. G01N 21/3504; G01N 33/00

(52) U.S. Cl. CPC G01N 21/3504; G01N 33/0047; G01N 2201/061; G01N 2201/066

(57) ABSTRACT

A gas sensor for sensing a presence of ethanol vapor in a cabin includes a source of infrared radiation, a first detector configured to receive infrared radiation from the source in a first region of the electromagnetic spectrum and a second detector for detecting a parameter, such as an amount of radiation received from the source in a second region of the electromagnetic spectrum, a temperature of the source and/or an amount of a second gas present in the cabin, affecting the amount of infrared radiation detected by the first detector. With this data, the presence of ethanol vapor in a cabin is established by an output of the gas sensor based on signals from both the first and second detectors.

### CROSS REFERENCE TO RELATED APPLICATION

The present application claims the benefit of U.S. Provisional Patent Application Ser. No. 61/968,537, filed on Mar. 21, 2014, entitled “Gas Sensor and Method of Sensing Presence of Ethanol Vapor in a Cabin.”

### BACKGROUND OF THE INVENTION

The present invention pertains to a gas sensor and, more particularly, to a gas sensor and method for detecting the presence of ethanol vapor within a cabin of a motor vehicle.

It is well known that drivers who have consumed alcohol (i.e., ethanol) pose a risk to themselves, their passengers, other vehicles and pedestrians. As a result, ways to detect whether someone has consumed alcohol have been developed, such as breath analyzers (also known as breathalyzers). In contrast to a breathalyzer, which requires that a user blow directly into it in order to detect the presence of ethanol in the user's breath, detecting the presence of ethanol vapor within a cabin of a motor vehicle does not require any affirmative action on the part of the driver or a passenger. Additionally, in situations where it is beneficial to know whether a passenger has consumed alcohol, such as on a job site or where the passenger is likely to be underage, detecting the presence of ethanol vapor within the vehicle cabin alerts a relevant party to this fact in a way that simply requiring the driver to blow into a breathalyzer does not.

However, because the cabin of a motor vehicle is relatively large, detecting the presence of ethanol vapor requires a sensor that can accurately detect low concentrations (e.g., 1 or 2 parts per million) of ethanol. To overcome this difficulty, previous attempts to provide an ethanol vapor sensor in the cabin of a motor vehicle have made use of collection technologies to gather ethanol into a more concentrated form. As a result, the measurements must be extrapolated and a delay is introduced. Therefore, there is considered to be a need in the art for a way to accurately and rapidly detect the presence of low concentrations of ethanol vapor in a cabin of a motor vehicle.

### SUMMARY OF THE INVENTION

The present invention is directed to a gas sensor and method for sensing a presence of ethanol vapor in a cabin (i.e., enclosed space), particularly a cabin of a motor vehicle. The sensor includes a source of infrared radiation, a first detector configured to receive and detect an amount of infrared radiation from the source and a second detector configured to detect another parameter affecting the amount of detected infrared radiation by the first detector in order to correct for changes in the amount of detected infrared radiation. More specifically, the first detector detects an amount of ethanol vapor that is present based on an amount of infrared radiation received from the source in a first region of the electromagnetic spectrum and an output of the gas sensor is based on signals from both the first and second detectors. In one preferred embodiment, the second detector also receives infrared radiation from the source and detects changes in infrared radiation emitted by the source based on an amount of radiation received from the source in a second region of the electromagnetic spectrum. In another preferred embodiment, a temperature detector or sensor measures a temperature of the source to correct for changes in infrared radiation received by the first detector due to changes in the temperature of the source. In still another preferred embodiment, the second detector detects an amount of a second gas that is present and, preferably employing a linear relationship based on a ratio of the second gas to ethanol vapor as a function of temperature, corrections are made for variations in infrared radiation emitted by the source due to temperature.

Additional objects, features and advantages of the present invention will become more readily apparent from the following detail description of preferred embodiments when taken in conjunction with the drawings wherein like reference numerals refer to corresponding parts in the several views.

### BRIEF DESCRIPTION OF THE DRAWINGS

FIG. 1 shows a gas sensor in a cabin of a motor vehicle in accordance with the present invention;

FIG. 2A is a perspective view of the gas sensor coupled to a tablet computer;

FIG. 2B is a side view of the gas sensor and tablet computer;

FIG. 3 shows a first embodiment of the gas sensor;

FIG. 4 shows a second embodiment of the gas sensor;

FIG. 5 shows a third embodiment of the gas sensor;

FIG. 6 shows a fourth embodiment of the gas sensor;

FIG. 7 is a graph showing changes in power due to changes in source temperature;

FIG. 8 is a graph showing the ratio of CO₂ to ethanol as a function of temperature;

FIG. 9 is a graph showing the ratio of ethanol to CO₂ as a function of temperature;

FIG. 10 is the resulting error curve when the CO₂ to ethanol ratio is assumed to be a constant;

FIG. 11 is the resulting error curve when the ethanol to CO₂ ratio is assumed to be a constant;

FIG. 12 shows a fifth embodiment of the gas sensor;

FIG. 13 is a graph showing the percent transmission of infrared radiation versus the angle of incidence for a taper in accordance with the present invention; and

FIG. 14 is a perspective view of the taper.

### DETAILED DESCRIPTION OF THE PREFERRED EMBODIMENTS

Detailed embodiments of the present invention are disclosed herein. However, it is to be understood that the disclosed embodiments are merely exemplary of the invention that may be embodied in various and alternative forms. The figures are not necessarily to scale, and some features may be exaggerated or minimized to show details of particular components. Therefore, specific structural and functional details disclosed herein are not to be interpreted as limiting, but merely as a representative basis for teaching one skilled in the art to variously employ the present invention.

The target performance for a gas sensor of the present invention is to reach single parts per million (ppm) levels of ethanol detection so that the sensor can function as a passive device that monitors background levels of ethanol that occur when inebriated occupants are present in a cabin of a motor vehicle. 70% of all drunk driving fatalities involve a driver with a blood alcohol content (BAC) greater than or equal to 0.08. This BAC corresponds to a breath alcohol content (BRAC) of roughly 200 ppm. An analysis of respiration rates was performed and a model of the expected ethanol concentrations in a typical midsize car cabin was generated for an occupant having a BRAC of 200 ppm and given 10 minutes of time. The results showed that greater than 85% of the modeled population would be detected as driving drunk if a 2 ppm limit of detection could be passively established. Accordingly, the sensor of the present invention is preferably configured to detect such concentrations of ethanol.

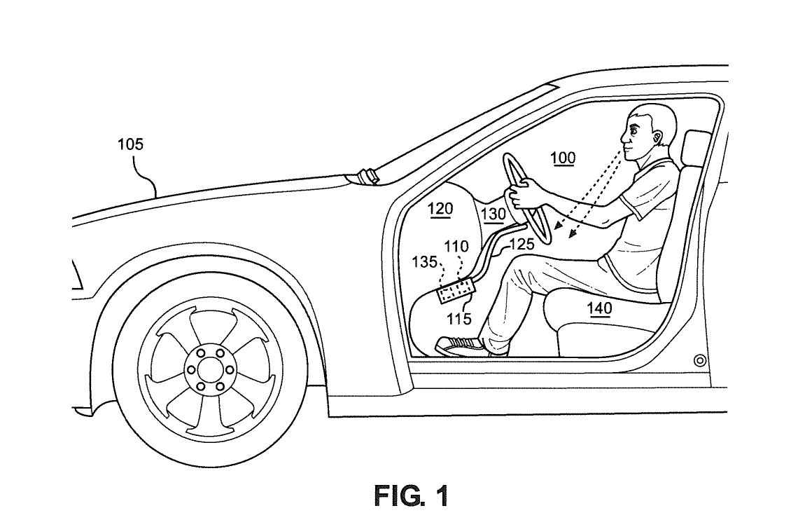

With initial reference to FIG. 1, there is shown a cabin 100 of a motor vehicle 105 in accordance with a preferred embodiment of the present invention. A gas sensor 110 is located in a sensor housing 115, which is coupled to a lower side of a dashboard 120 of vehicle 105 with tubing 125 running along a steering column 130. As a result, sensor 110 is mounted out of the way and also, optionally, out of sight. As the concentration of ethanol vapor at the location of sensor 110 is likely to be lower, tubing 125 is provided so that air can be drawn, by a fan 135 included in housing 115, from an area where the concentration of ethanol vapor is likely to be higher (i.e., nearer to the driver's face). In addition to mounting sensor 110 underneath dashboard 120, it should be readily apparent that sensor 110 can be located in a variety of other locations throughout cabin 100 with or without tubing 125. For example, in one embodiment, sensor 110 is located on top of dashboard 120. In another embodiment, sensor 110 is enclosed by dashboard 120 or some other structure, and tubing 125 runs from inside dashboard 120 to an area outside dashboard 120. Alternatively, sensor 110 is located beneath a driver seat 140, and tubing runs along a side of driver seat 140. Although motor vehicle 105 is depicted as an automobile in FIG. 1, it should be noted that sensor 110 can be used in any vehicle where it would be desirable to detect the presence of ethanol vapor in a cabin, such as an airplane, a train or a boat.

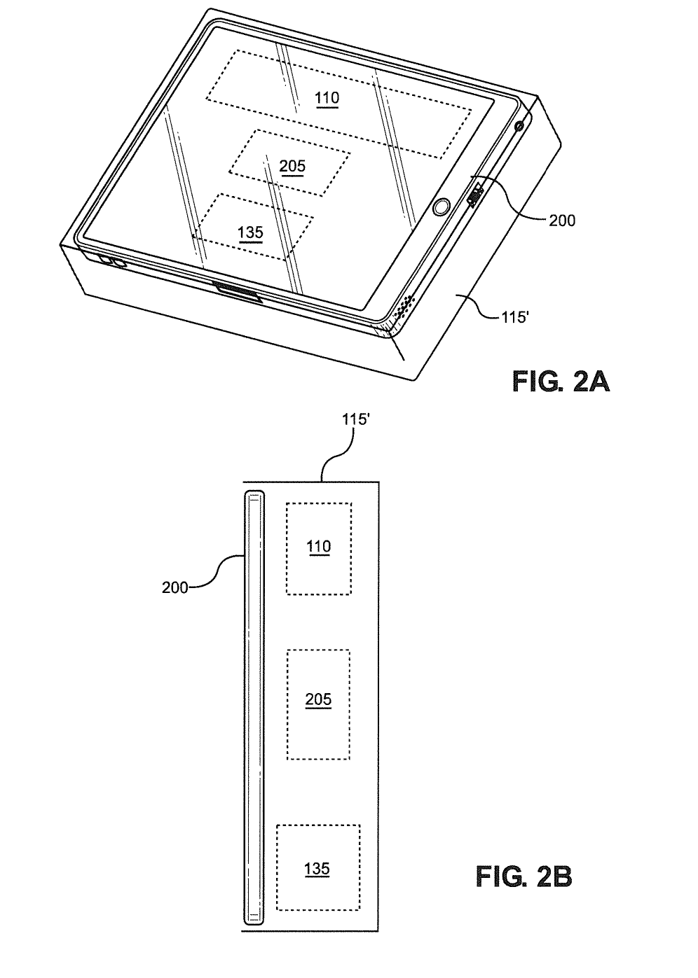

FIGS. 2A and 2B show an embodiment where sensor 110 is located in a sensor housing 115′ coupled to a tablet computer 200. Housing 115′ also includes data acquisition electronics 205, such as a digital acquisition board and a power conditioning/pulse control board, and fan 135 for establishing a flow of air in sensor 110 and housing 115′. As a result, software on tablet computer 200 interacts with data acquisition electronics 205 to facilitate analysis, display and transmission of data provided by sensor 110. In one example, tablet computer 200 displays a real-time signal indicative of the amount of ethanol vapor detected within cabin 100. Although reference is made to fan 135, one or more low-power, miniature fans are used in a preferred embodiment. The arrangement described above is especially beneficial when sensor 110 is coupled to an upper portion of dashboard 120, for example, such that tablet computer 200 is visible. However, tablet computer 200 need not be visible in an installed position.

In a preferred embodiment, once data has been collected by sensor 110, the data is wirelessly transmitted to a remote location for use in real-time monitoring of driver risk. In addition to data regarding the presence of ethanol vapor in cabin 100, the time and location of vehicle 105 (provided by GPS, for example) are preferably included. The transmission can be accomplished by connecting sensor 110 to tablet computer 200 (described above), a mobile phone, a communication system integrated in motor vehicle 105 or any other wireless transmission system known in the art. Alternatively, a wireless communication system can be included in sensor 110 or housing 115. The data can be processed prior to transmission either by sensor 110 or by the device to which sensor 110 is connected, or the data can be processed after transmission by the system that receives the wireless data. In one embodiment, the data is sent to a secure website where it can be viewed by a concerned party, such as a parent, a foreman, a fleet manager or an insurance company. Although the data can indicate the presence of ethanol vapor within cabin 100, and hence the consumption of alcohol, it might not distinguish between consumption of alcohol by a driver and consumption of alcohol by a passenger. However, simply knowing that someone in vehicle 105 has consumed alcohol is useful in certain situations. For example, a foreman is likely to be concerned whether anyone in a work vehicle has consumed alcohol. Similarly, a parent of an underage driver may want to know whether one of his child's passengers has consumed alcohol. This system is able to provide that information in real-time to a user remote from vehicle 105. In addition, it should be noted that the data does not need to be transmitted in real-time to a remote location. In an alternative embodiment, the data is stored in sensor 110 or the device connected to sensor 110 (e.g., tablet computer 200 or data storage on vehicle 105) so that it can be viewed or downloaded at a later time. Also, the data might be transmitted wirelessly, but less frequently, such as once a day.

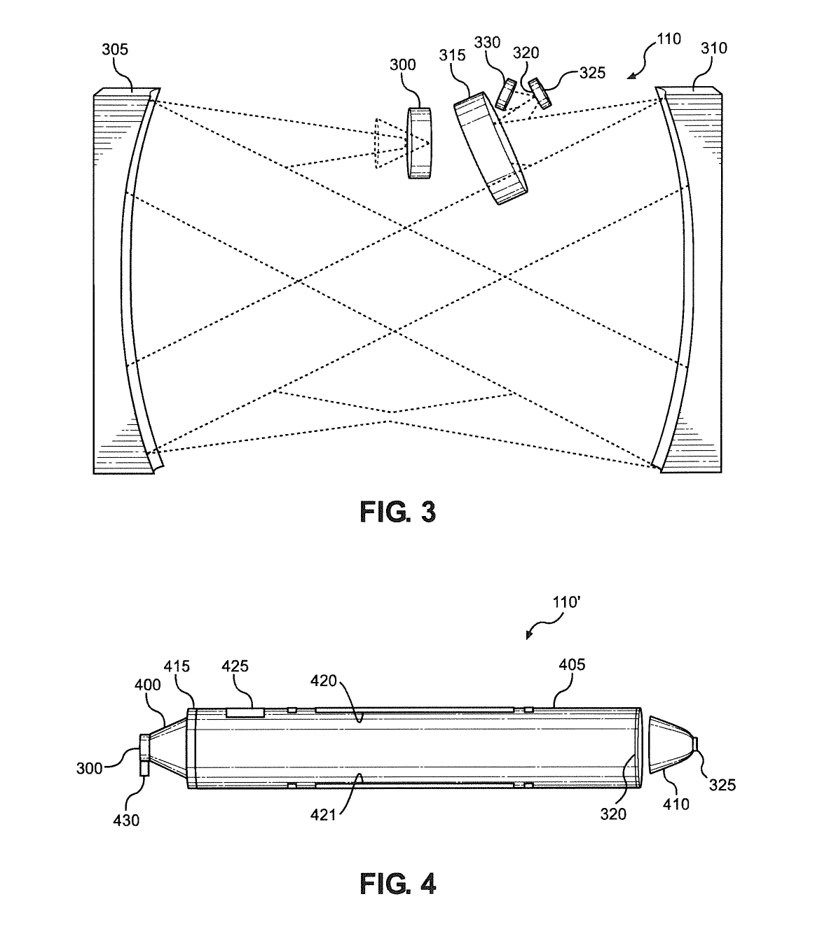

Turning now to FIG. 3, a first embodiment of sensor 110 is shown. In general, an infrared (IR) signal is emitted by sensor 110 and continuously monitored. The presence of ethanol vapor causes absorption of the IR signal, which is detected by sensor 110. Sensor 110 includes a thermal IR source 300, two spherical mirrors 305, 310, a turning or adjustable mirror 315, a filter 320, a first thermopile detector 325 and a second thermopile detector 330. IR radiation created by source 300 travels from source 300 to mirror 305, from mirror 305 to mirror 310, from mirror 310 back to mirror 305, from mirror 305 back to mirror 310 and then from mirror 310 to turning mirror 315. Turning mirror 315 collects this energy and focuses it on filter 320 and first detector 325. Filter 320 allows radiation in the 9-10 μm region of the spectrum to pass to first detector 325, with this region being selected as the main absorption band for ethanol. Radiation that does not fall within this band is reflected to second thermopile detector 330, which operates over the 8-14 μm region of the spectrum. As a result, two signal channels are created, i.e., an ethanol channel from first detector 325 and a power-monitoring channel from second detector 330. The two channels are used in a ratio to determine absorption in the ethanol band, which eliminates the main problems posed by using a single detector, namely, sensitivity to power fluctuations in source 300 or optical integrity degradation of mirrors 305, 310 over time. By scaling and dividing the power-monitoring channel signal into the ethanol channel signal, only the absorption due to the presence of ethanol is measured. This measurement is sensitive enough to detect concentrations of ethanol on the order of 1 ppm and is predominantly limited by electronic noise rather than spectral noise.

Additionally, this optical design provides a common path to detectors 325, 330 so that scattering due to dust or film development on mirrors 305, 310 is mutually accounted for on each channel. Furthermore, the X-shaped, folded optical path allows for a relatively long path length (preferably approximately 400 mm) in a relatively small area (roughly 76 mm×89 mm×38 mm in one embodiment). This path length represents a balance between several design parameters. A long path length provides greater sensitivity to low concentrations of molecules, but also reduces signal strengths toward the electronic noise floor, which is undesirable. Also, it is beneficial to minimize the package size to provide a compact layout for use in cabin 100 of motor vehicle 105.

In one particular embodiment, mirrors 305, 310 are approximately 20 mm in thickness and 50 mm in height. Mirrors 305, 310 are sliced from larger 50 mm diameter circular mirrors and are gold-coated reflectors with high reflectivity in the spectral region of interest (i.e., 9-10 μm). The radii of mirrors 305, 310 are 38 mm, and the radius of mirror 315 is 30 mm. Mirror 315 is a stock part made by Edmund Optics (#46-234). Source 300 is a silicon membrane heated resistively to greater than 650° C. to create sufficient IR radiation for emission. Source 300 is driven using a square wave current at 6.5 V and approximately 140 mA with a 50% duty cycle. This allows the system to be AC coupled and mathematically filtered at a drive frequency of 8 Hz. This design results in 0.5 V signal strengths with 100 μV precision using minimal biases of 5 V for detectors 325, 330, which use application-specific integrated circuit (ASIC) amplifier technology to ensure low noise operation and reduced thermal energy. A 24-bit data acquisition system (not shown) is used with a differential amplifier or ratioing circuit for the manipulation of the outputs of the two channels.

FIG. 4 shows a gas sensor 110′ according to a second embodiment of the present invention. As in the first embodiment, sensor 110′ includes source 300, filter 320 and first detector 325. Source 300 emits IR radiation into a first parabolic reflector 400, which is preferably nickel-electroformed with a protected aluminum coating. First parabolic reflector 400 directs the IR radiation into an aluminum tube 405, the inside surface of which is preferably electropolished to provide a mirror-like reflective surface. Accordingly, tube 405 acts as a light conduit, channeling the energy to filter 320 which, as above, allows transmission of radiation in the 9-10 μm region of the spectrum. As a result, radiation passing through filter 320 is restricted to the band coincident with the major absorption line of ethanol, and the presence of ethanol in tube 405 will modulate the signal strength of the IR radiation passing through filter 320. The radiation is then collected by a second parabolic reflector 410, constructed in the same manner as first parabolic reflector 400, which focuses the energy to detector 325 so that an ethanol-sensing channel is established.

Sensor 110′ also includes a spacer 415 in tube 405 that securely holds first parabolic reflector 400 against source 300 to maintain their relative alignment. Spacer 415 has radial grooves (not shown) along one face to allow air to circulate between the inside and outside of tube 405. Additionally, electronic boards 420, 421 are provided to facilitate data acquisition and processing. Fan 135 (discussed above but not shown in FIG. 4), which is preferably a low-profile, blower fan, is provided to create an air flow so that tube 405 is filled with air, thus allowing for maximum sensitivity of sensor 110′. The path length of this design is preferably approximately 150 mm in order to provide a sufficient path length for the absorption of the IR radiation by ethanol molecules such that the strength of the signal is affected. The design also confines the radiation to predominantly + or −10 degrees of incidence on filter 320 to maintain desired filtering performance.

In one particular embodiment, source 300 is an IR carbon membrane source made by Hawkeye Technologies (IR-50), which is used as a modulatable emitter of pulses to allow AC coupling of the signals. Source 300 is 1.5 mm×1.5 mm, and, when driven as a 4 Hz square wave with greater than 90% modulation, is approximately 1 W and 1020° C. Parabolic reflectors 400, 410 have focal lengths of 1.375 mm and are approximately 18.4 mm in diameter at the largest end. The reflectivity of the inside surface of tube 405 is preferably greater than 85%. Filter 320 is made from a germanium substrate, but other substrates, such as silicon, can also be used. Filter 320 is centered at 9.466 μm with 85% transmission between 9.1 and 9.65 μm. Detector 325 is a thermopile detector made by Heimann Sensor (HCM-C22), which has an integrated gain amplifier and thermistor in an ASIC solid-state package with a cover glass filter that restricts radiation to the 8-14 μm region.

In addition to the ethanol-sensing channel described above, sensor 110′ includes another detection channel along the side of tube 405 for carbon dioxide (CO₂). This is accomplished through the use of CO₂ detector 425, which allows the presence of exhaled breath in cabin 100 to be monitored. The amount of CO₂ in cabin 100 is commensurate with the amount of ethanol, although the rates of build-up may vary between the two due to certain factors, such as an occupant's metabolism and level of inebriation. However, the derivative of change is correlated between the two as CO₂ and ethanol emitted by an occupant of vehicle 105 follow the same dilution dynamics in cabin 100. As a result, ethanol absorption signals should not increase without commensurate increases in CO₂, although CO₂ can increase without an increase in ethanol absorption when the driver (and any passengers) are sober. Research has shown that levels of exhaled CO₂ and ethanol are relatively constant over a period of at least 9 to 82 minutes after consumption of alcohol. For a given period of time on the order of 10 minutes, the rate of ethanol exhalation varies only slowly. Accordingly, during that period, absorption increases in the ethanol-sensing channel, without increases in CO₂ absorption, represent interference from other gases. The present invention takes advantage of this by simultaneously monitoring the presence of CO₂ and ethanol and their relative changes over time in cabin 100 of motor vehicle 105. Sensor 110′ (through the use of a controller on electronic boards 420, 421, for example) determines the presence of ethanol vapor in cabin 100 based on an amount of increased absorption on the ethanol-sensing channel, and then determines a mathematical correlation between the ethanol-sensing channel and the CO₂ channel. A strong correlation between the channels over a 10-minute period indicates the presence of ethanol. Weak or no correlation indicates that no ethanol is present or that it is masked by other gases. The trend over the 10-minute period is a descending trace representing the increase in absorption.

In a further aspect of this invention, sensor 110′ includes a detector 430, with a built-in thermistor, which is coupled to source 300 for monitoring the temperature of source 300. The temperature of source 300 can vary by very small amounts, yet even this variance can sway the readings of the ethanol and CO₂ channels on the order of one or more parts per million. To account for this, the temperature of source 300 is monitored by detector 430 and the readings of the signal channels are adjusted accordingly. Source 300 acts as a blackbody radiator and behaves according to Planck's Law, which is embodied in the following equations:

$$\begin{matrix}{B}_{v} ( T ) = \frac{2 {hv}^{3}}{{c}^{2}} \frac{1}{{e}^{\frac{hv}{{k}_{B} T}} - 1} \\ or & Equation 1 \\ {B}_{λ} ( T ) = \frac{2 {hc}^{2}}{{λ}^{5}} \frac{1}{{e}^{\frac{hc}{λ {k}_{B} T}} - 1} & Equation 2\end{matrix}$$

Where B is spectral radiance, T is absolute temperature, k_{B} is Boltzmann's constant, h is Planck's constant and c is the speed of light. As source 300 heats up during startup, more energy arrives per second in each of the ethanol and CO₂ channels. If a cooling event occurs that drops the temperature of source 300, less energy will arrive. In either case, this is not representative of the signal itself (i.e., the amount of ethanol or CO₂), but could be interpreted as absorption loss or gain if not corrected for. For example, in one embodiment, a signal indicative of 1 ppm of ethanol only modulates the energy in the ethanol-sensing channel by a small amount (from 100% to 99.9943%), and this change could easily be masked or amplified by an increase or decrease in the temperature of source 300. Therefore, it is preferable that the temperature of source 300 is known to at least an equal degree of precision (e.g., 0.033 degrees Kelvin) to enable this level of detection. Additionally, ethanol detector 325 and CO₂ detector 425 preferably include one or more built-in thermistors so that correction factors can be calculated (by a controller on electronic boards 420, 421, for example) in order to account for responsivity changes that occur with temperature shifts. In other words, the responses of detectors 325, 425 to IR radiation received from source 300 are affected by temperature, and, therefore, these responses are preferably adjusted to account for changes in the temperature of detectors 325, 425.

In one particular embodiment, detector 430 is an ASIC chip, and it is coupled to a TO-5 can of source 300 by a high thermal conductivity epoxy (e.g., Master Bond Polymer System EP30AN-1). As a result, heat is transferred from source 300 to the thermistor of detector 430 in 1-2 seconds. The thermistor is a fast response device that allows multiple independent measurements to be made on the order of 10 samples per second or greater. For the HCM-C22 chip from Heimann Sensor, the responsivity of the embedded thermistor is on the order of 15 mV per degree Celsius. For experimental noise values of 100 μV, the temperature resolution is 0.007° C. for a single read. The combination of fast sampling and high resolution can be combined to provide many reads, which can be averaged to obtain an even more precise assessment of temperature changes.

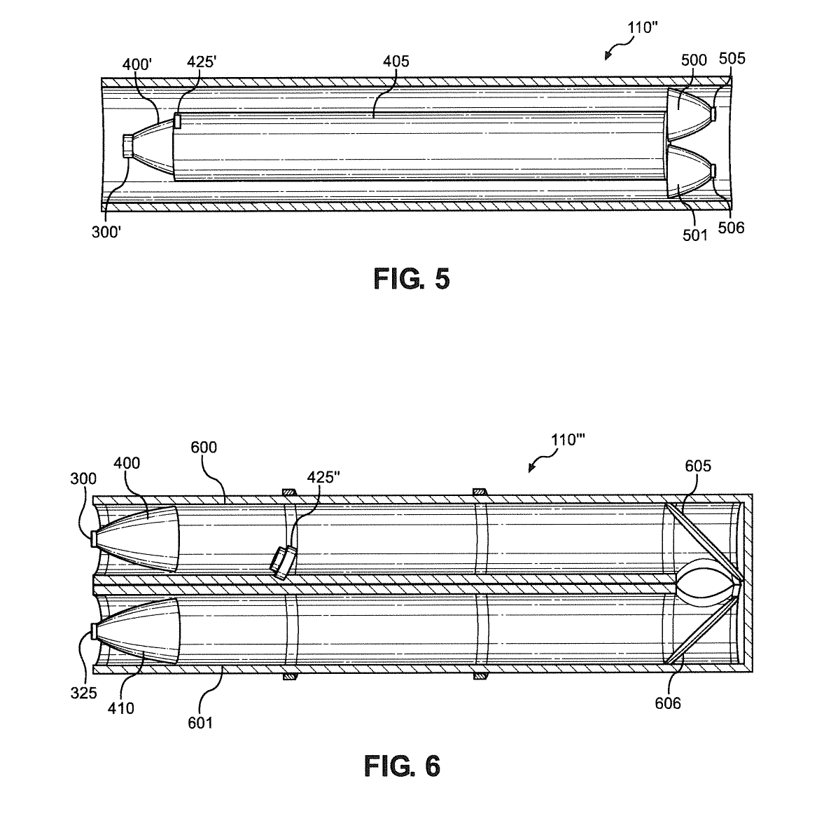

With reference now to FIG. 5, a third embodiment of the present invention is shown. In place of second parabolic reflector 410 and first detector 325, gas sensor 110″ includes two parabolic reflectors 500, 501 and two detectors 505, 506. Additionally, a larger source 300′ (such as the IR-70 made by Hawkeye Technologies) is used to provide more coupled power to each of detectors 505, 506 to compensate for the splitting of the field. As a result of this design, another detection channel is provided in a region of the spectrum adjacent to the ethanol absorption band. For example, in a preferred embodiment, a band centered at 10.4 μm is used, which is close enough to an ethanol band centered at 9.4 μm that the power emitted by source 300′ can be tracked. In particular, temperature variations in source 300′ are compensated for by monitoring changes in the second band. In other words, any change in the signal received by detector 506 (corresponding to the second detection channel) would indicate a change in the radiation emission of source 300′, while a change in the signal received by detector 505 (corresponding to the first, ethanol detection channel) could indicate either the presence of ethanol vapor or a change in radiation emission of source 300′. By including both signal channels, changes due to temperature variations of source 300′ can be isolated from changes due to the presence of ethanol. Accordingly, a thermistor is not coupled to source 300′ in this embodiment since the second detection channel provides the same general functionality in terms of allowing the algorithm to account for thermal drift.

This design also avoids the use of folded ray paths or expensive IR crystal optics, such as dichroic beam splitters with large areas, while providing a strong signal to both channels. Although the overall diameter of the system increases to accommodate both parabolic reflectors 500, 501, this creates more room for electronics to be integrated, which reduces the cost of miniaturizing the electronics (such as electronic boards 420, 421, which are not shown in FIG. 5). Specifically, in one embodiment, the overall sensor 110″ is now 38 mm as compared with 30 mm for sensor 110′. Alternatively, in another configuration, parabolic reflectors 500, 501 are electroformed as a single optic at a smaller diameter (not shown) by eliminating the unused wing portions of each parabolic reflector 500, 501. In other words, the top and bottom portions (relative to FIG. 5) of each parabolic reflector 500, 501 are eliminated to reduce the diameter of the combined part. Forming parabolic reflectors 500, 501 as a single part also facilitates alignment and serves to maintain a split ratio during operation, thus reducing amplitude noise that would arise due to vibration of the parts. Similarly, in such an embodiment, a single detector board with two ASIC chips (not shown) can be used, the chips being mounted such that they match the positions of the outputs of parabolic reflectors 500, 501. As a result, this design reduces the number of alignment operations to one at each end of tube 405.

As in the second embodiment, sensor 110″ further includes a CO₂ detector 425′ on its own electronic board, which is shown placed at the junction of a first parabolic reflector 400′ and tube 405. Parabolic reflector 400′ has a slightly longer dimension in this embodiment to provide better coupling to larger source 300′. Preferably, source 300′ is used without an external can in order to provide access to the emitter plane for better coupling of the signal. Optionally, source 300′ is coupled to a short, reflective tube (not shown) to provide homogenization of the spatial output of source 300′ prior to being imaged by parabolic reflector 400′. A homogenizer with high reflectivity mixes the varying spatial pattern of emission of source 300′. Carbon membrane sources can have non-uniform emission patterns due to their flexure under thermal stress during pulsed operation. This can lead to variability in the coupled power thus requiring longer integration times to control the effect. By using a pre-mixing stage, spatial variability is reduced without greatly affecting signal strength.

FIG. 6 shows a fourth embodiment of the present invention that allows for the detection of ethanol at the parts per billion (ppb) level. Additionally, a mathematical relationship between two signal channels of detection is used to account for variations in source 300. Gas sensor 110′″ now includes two tubes 600, 601 and two mirrors 605, 606 arranged at 45 degree angles, which relay IR radiation coming from source 300 in upper tube 600 to ethanol detector 325 in lower tube 601. This extends the path length roughly three times such that sensitivity is increased to below 1 ppm into the ppb range. A CO₂ detector 425″ is shown placed in tube 600 a short distance from source 300, which is located at the focus of first parabolic reflector 400, while ethanol detector 325 is located at the focus of second parabolic reflector 410. In a preferred embodiment, this dual-tube configuration is roughly 25.4 cm long and 6.35 cm wide.

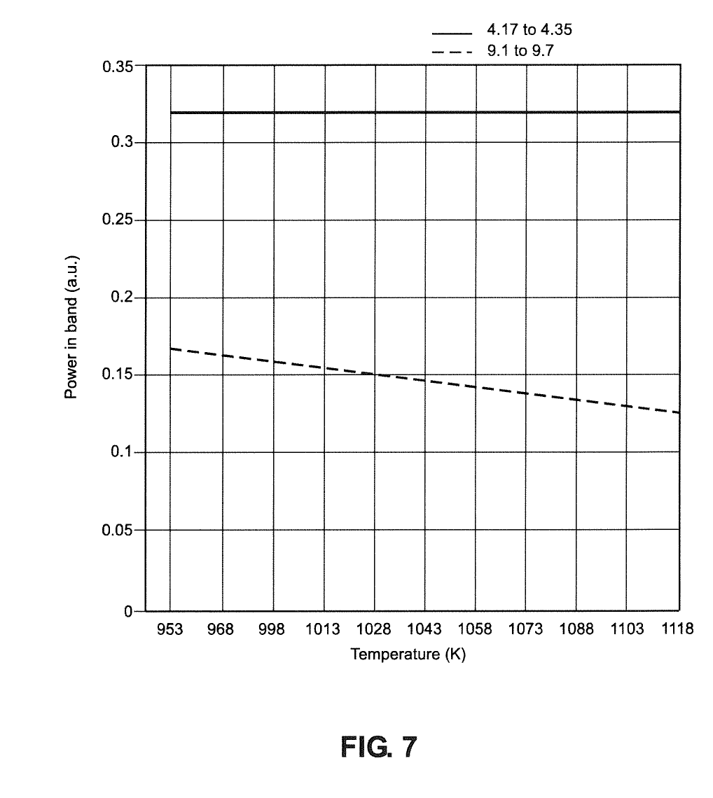

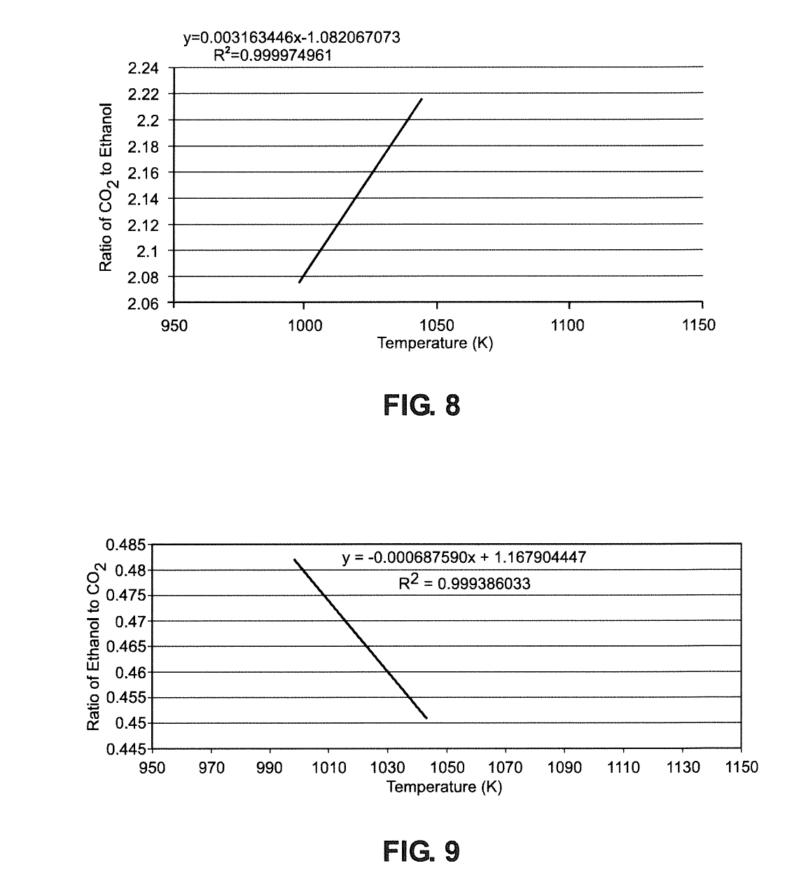

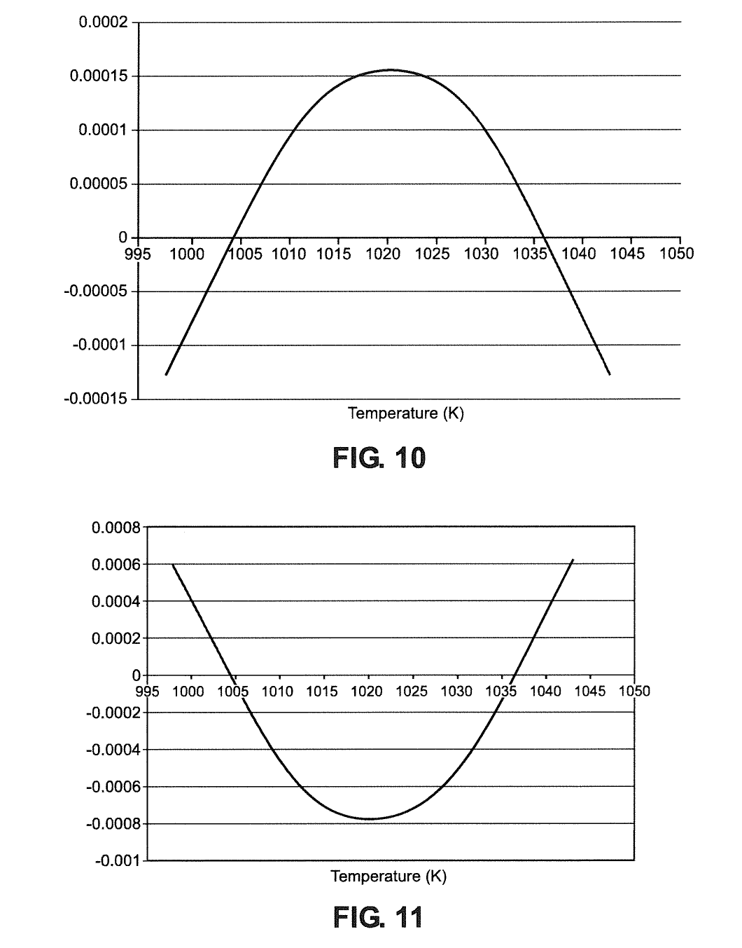

As discussed above, it is beneficial to monitor and correct for variations in source 300. This particular embodiment forgoes the use of a thermistor or an adjacent detection channel and instead uses a ratio of the signals from the two signal channels (ethanol and CO₂) such that variations in the intensity of IR radiation emitted by source 300 do not prevent sensor 110′ from detecting small changes in the signal channels. Source 300 is modeled as a blackbody radiator using Equation 1, included above. The equation calculates the energy per unit time (or the power) radiated by a blackbody at temperature T. Source 300 was modeled using a number of temperatures to simulate the variation of the blackbody with temperature, and the energy in the detection bands of interest (4.17 to 4.35 μm for CO₂ and 9.1 to 9.7 μm for ethanol) was calculated. FIG. 7 shows the variation in power in each channel with temperature. When taking the ratio of the powers in the channels, temperature variations change the power in each channel by different degrees and thereby influence the ratio such that ethanol could falsely be read as being present or absent. It was found that taking the ratio of the powers in these two channels, over a nominal temperature range of operation, yields the curves shown in FIGS. 8 and 9. FIG. 8 shows the ratio of CO₂ to ethanol as a function of temperature and the resulting linear fit of that relation, which shows a high degree of linearity. Similarly, FIG. 9 shows the ratio of ethanol to CO₂ as a function of temperature and the resulting linear fit. The error curves resulting from these linear relationships are shown in FIGS. 10 and 11. In other words, FIGS. 10 and 11 show the expected change due to variations in temperature if each relationship is assumed to be a constant. As the R² value is greater and the error smaller for the relationship shown in FIGS. 8 and 10 (CO₂/ethanol), this is the preferred embodiment.

As a result of the above, the ratio of the source power in the CO₂ and ethanol channels can be treated as a constant of the first order over a reasonable range of operating temperatures. The derivative of the ratio of the signal of the channels allows the term containing the incident power (I in Equations 3 and 4, include below) to be treated as a constant. This constant is the slope of the linear fit in FIG. 8. Accordingly, the temperature change of source 300 is treated as if it were the time dependent change of the ratio of CO₂ to ethanol and this is found to be a constant to high accuracy over the range of 998 to 1043 Kelvin (i.e., + or −25 degrees about the nominal temperature of source 300). Equations for each signal channel are shown below, with each equation representing a signal voltage that the relevant detector produces:

V(eth)=G(t1)*Tₑₜₕ*Iₑₜₕ*e^{(-α*L1*C1)} Equation 3:

V(CO₂)=G(t2)*T_{CO}_{₂}*I_{CO}_{₂}*e^{(-β*L2*C2)} Equation 4:

Wherein α is the absorbance in ppm-meter for ethanol; β is the absorbance in ppm-meter for CO₂; L1 is the path length for the ethanol channel; L2 is the path length for the CO₂ channel; C1 is the concentration in ppm for ethanol; C2 is the concentration in ppm for CO₂; G(t1) is the responsivity and electrical gain for ethanol detector 325; G(t2) is the responsivity and electrical gain for CO₂ detector 425″, which may be at a different temperature than ethanol detector 325; Iₑₜₕ is the energy incident on ethanol detector 325 in watts; and I_{CO2} is the energy incident on CO₂ detector 425″ in watts.

The G terms (i.e., G(t1) and G(t2)) are time and temperature dependent, and they are corrected for and held constant by the thermistors built into detectors 325, 425″, as described in connection with the second embodiment. The absorbance and path lengths are constant for both channels for any measurement reading. The transmission values (i.e., Tₑₜₕ and T_{CO2}) are slowly varying values that are related to the gradual degradation of the optical system over months or years of time and can be considered constant as well for any interrogation period of interest, which is approximately 5 to 10 minutes. By taking the time-dependent derivative of the ratio of Equation 4 to Equation 3, the time derivative of the source intensity ratio (I) can be considered as constant based on the linearity demonstrated in FIG. 8. The expected change due to 1 ppm for sensor 110′″ is 9×10⁻⁵ units. The residual error of using this approximation is less than or equal to 1 ppm of ethanol change in signal over + or −10 degrees with less than 0.00005 units of error. As a result, as long as the temperature drift of source 300 is less than 10 degrees over any 10-minute interrogation period, the error in reading the ppm level is less than 0.5 ppm. Preferably, the algorithm baselines itself in each 10-minute period of operation so that this full 10 degree tolerance is available. Gradual drift in temperature on a long timescale is negated by this re-baselining approach, which allows the derivative of the ratio approach to be dominated by changes in ethanol and CO₂ concentrations rather than by source fluctuations.

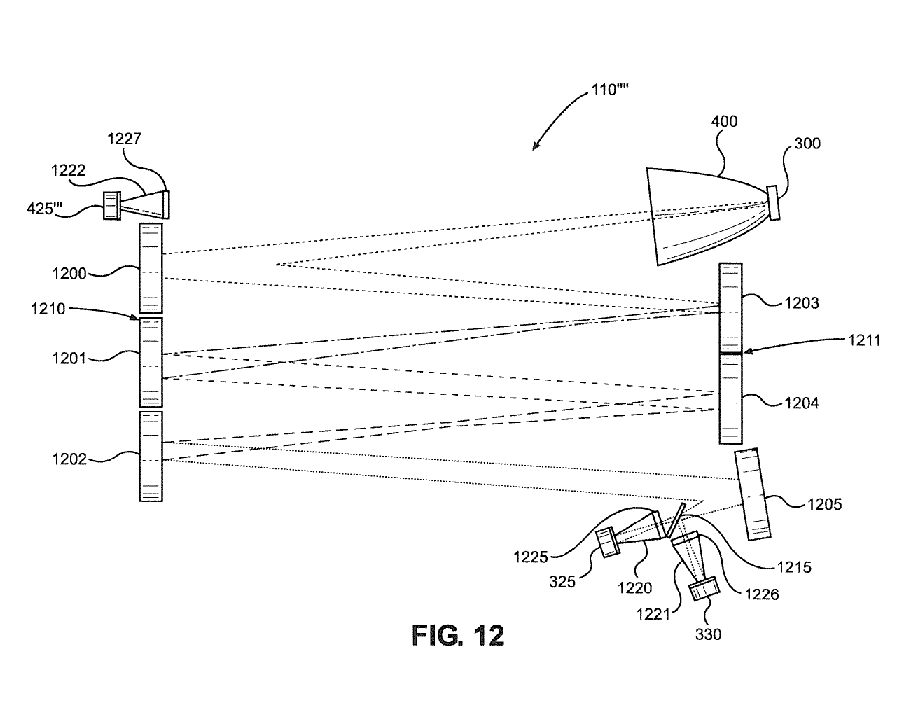

Turning to FIG. 12, there is shown a fifth embodiment of the present invention. In this embodiment, the path length is extended, such as on the order of 1 meter, to increase sensitivity, while a high-throughput, efficient optical design is maintained through the use of spherical mirrors and tapers. This enables detection at the ppb level for ethanol, as well as other gases of interest, using low-cost components. In one preferred embodiment, the path length is 945 mm and the package is 132 mm×215 mm×50 mm, thereby providing a relatively long path in a relatively small volume. As in the other embodiments discussed above, a sensor 110″″ includes source 300, first detector 325, second detector 330 and first parabolic reflector 400. Sensor 110″″ also includes three reflectors or mirrors 1200, 1201, 1202 on the left and three reflectors or mirrors 1203, 1204, 1205 on the right (relative to FIG. 12). Although mirrors 1200-1205 appear to be discrete, mirrors 1200-1205 are preferably formed as part of two nickel-plated surfaces that combine mirrors 1200-1205 into two reflectors 1210, 1211 having spherical surfaces at each position. The IR radiation from source 300 is collimated by parabolic reflector 400 and directed to the series of reflective spherical surfaces (i.e., mirrors 1200-1205) formed on reflectors 1210, 1211. Specifically, IR radiation travels from source 300 to mirror 1200, from mirror 1200 to mirror 1201, from mirror 1201 to mirror 1202, from mirror 1202 to mirror 1203, from mirror 1203 to mirror 1204 and from mirror 1204 to mirror 1205. Mirror 1205 directs the IR radiation to a splitter 1215, which sends the radiation to both detector 325 and detector 330. Additionally, a CO₂ detector 425′″ is provided near the top of sensor 110″″ (relative to FIG. 12) with a direct path to source 300. As a result, three detection channels are provided: an ethanol channel using detector 325; a power-monitoring channel using detector 330; and a CO₂ channel using CO₂ detector 425′″. The nature of these channels should be readily apparent from the discussion of the other embodiments. Therefore, this discussion will not be repeated here. In an alternative embodiment, splitter 1215 and detector 330 are omitted and fluctuations in source 300 are accounted for by monitoring the temperature of source 300 or using the calculations described in connection with the fourth embodiment.

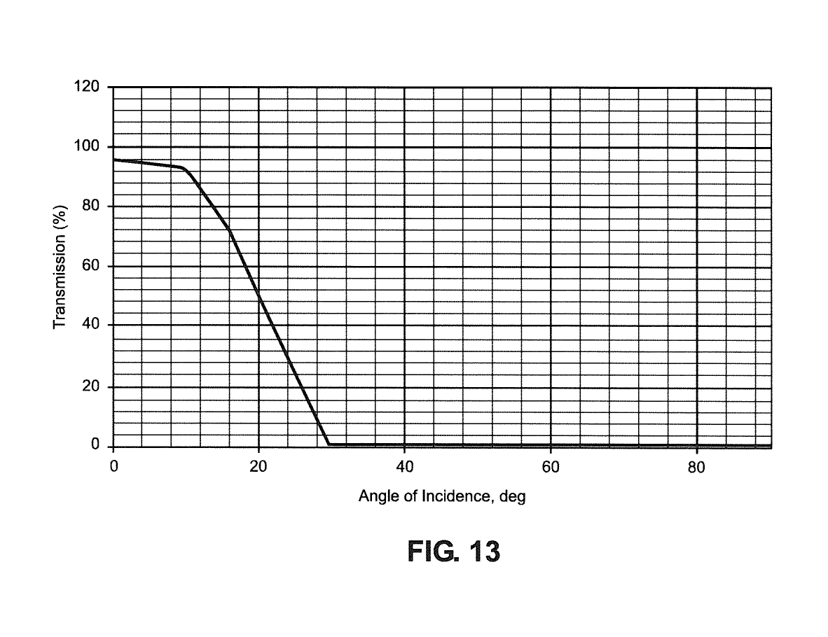

In a preferred embodiment, sensor 110″″ includes tapers 1220, 1221, 1222 coupled to detectors 325, 330, 425′″, respectively. Tapers 1220, 1221, 1222 increase the throughput to detectors 325, 330, 425′″ (from 5.8% to greater than 10% in one embodiment) and also provide noise reduction by rejecting unwanted reflections of IR radiation. Specifically, tapers 1220, 1221, 1222 reflect away those ray paths that are outside the primary ray path provided by source 300, mirrors 1200-1205 and splitter 1215 (or simply source 300 in the case of detector 425′″). Otherwise, any IR radiation that leaks outside this ray path acts as random noise and modulates the signal received by detectors 325, 330, 425′″, which is undesirable as it can cause inaccurate readings. Tapers 1220, 1221, 1222 reduce this effect by restricting the angles at which IR radiation can reach detectors 325, 330, 425′″ to those angles that correspond to the desired axis of the optical system. FIG. 13 shows the percent of IR radiation transmitted based on the angle from which the IR radiation is approaching (i.e., the angle of incidence). As should be readily apparent from FIG. 13, tapers 1220, 1221, 1222 are effective at limiting IR radiation coming from outside the primary optical path and thereby reduce noise.

Another benefit of tapers 1220, 1221, 1222, and the overall design, is that selective filters 1225, 1226, 1227 for the radiation bands of interest are located at the entrances of tapers 1220, 1221, 1222 where the incident angles are less steep. This minimizes the detuning of the filter function and its center wavelength. Additionally, it minimizes the area of expensive, thin-film-coated filters 1225, 1226, 1227 and thereby reduces costs while still allowing operation in a high-throughput condition with high signal-to-noise ratios. IR radiation exiting tapers 1220, 1221, 1222 at detectors 325, 330, 425′″ is more steeply incident, but the selective action of filters 1225, 1226, 1227 has already taken place, which allows for the collection of more IR radiation.

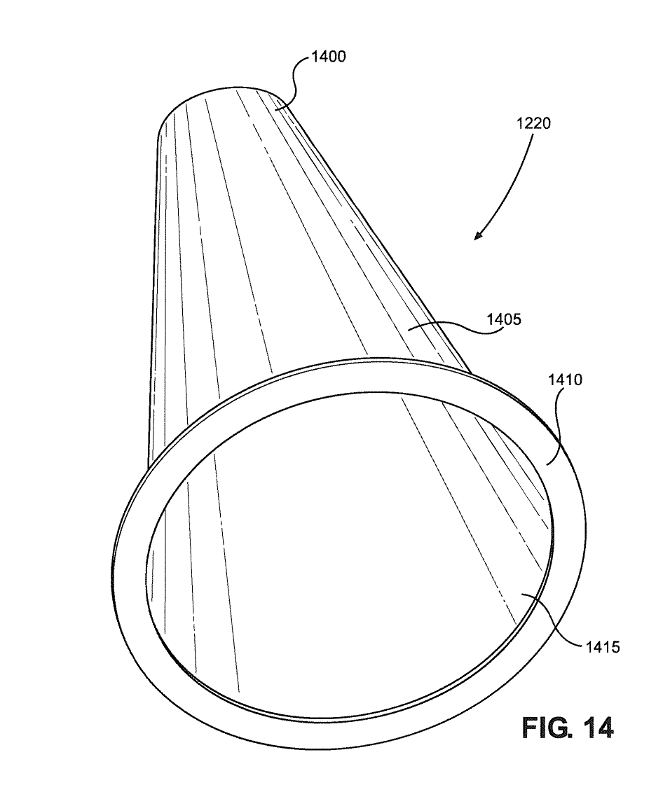

FIG. 14 shows a more detailed view of taper 1220, although tapers 1221, 1222 can be constructed in the same manner. In one embodiment, taper 1220 has a length of 12.1 mm, an inner diameter of 2.6 mm at a first end 1400 and an inner diameter of 7.0 mm at a second end 1405. Additionally, the diameter of fin 1410 is 8.2 mm while the thickness of fin 1410 is between 0.1 and 0.5 mm. An interior 1415 of taper 1220 is coated with aluminum and silicon dioxide (SiO₂) to provide an optical finish. High reflectance coatings (preferably having 97% reflectivity) are used for the various surfaces of mirrors 1200-1205 and tapers 1220, 1221, 1222 to provide a high efficiency channel of interrogation for sensor 110″″. In one particular embodiment, mirrors 1200-1204 have radii of curvature of −150 or −200 mm, while mirror 1205 has a radius of curvature of −50 mm. Mirrors 1200-1205 can be circular or rectangular in their cross-section. Mirrors 1200, 1201, 1202 are co-planar to make it easier to manufacture the set as a single, nickel-plated sheet (i.e., reflector 1210). Mirrors 1203, 1204, 1205 are formed in the same manner, with mirror 1205 tilted relative to mirrors 1203, 1204 by approximately 3 degrees. Forming mirrors 1200, 1201, 1202 and mirrors 1203, 1204, 1205 as single components simplifies their placement. In other words, the precision alignment of the parts occurs during fabrication rather during assembly, which reduces labor costs.

When compared to the prior art, the advantages of the fifth embodiment should be readily apparent. For example, U.S. Pat. No. 5,009,493 to Koch et al. (hereinafter “Koch”) discloses a three-mirror design. If scaled to offer the same path length (i.e., roughly 1 meter) and using the same source and detector sizes for both the Koch design and the fifth embodiment of the present invention, an analysis revealed that the Koch design only offers less than 1.8% transmission of the source IR radiation to the detector. In contrast, the fifth embodiment provides roughly 10% transmission. In other words, the fifth embodiment is five times more efficient in its use of IR radiation. This results in lower power consumption, which is beneficial in a portable application; higher signal-to-noise ratios since more IR radiation arrives at the detector; and a less complex fabrication procedure due to the use of spherical surfaces rather than the toroidal ellipsoidal mirrors required by Koch. In another example, U.S. Pat. No. 7,449,694 to Yi et al. (hereinafter “Yi”) discloses a two-mirror design with intermediate focal planes. As compared to the fifth embodiment of the present invention, Yi has several disadvantages. First, the intermediate focal planes increase the aberrations present in the system and limit the amount of IR radiation that can reach the detector. Second, Yi's design collects much more stray noise due to the use of the enclosed two-mirror structure without an angularly selective element (e.g., a taper). Third, the amount of IR radiation that can be transmitted through the two-mirror chamber is limited due to the crossing of the beams (i.e., the overlapping of the beams). In particular, the design shown in FIG. 21 of Yi will capture less than 2.1% of the source IR radiation passing through the system, and the final value will actually be less than this due to the aberrational losses cited above. Therefore, the 10% transmission provided by the fifth embodiment is clearly superior to the design of Yi.

Based on the above, it should be readily apparent that the present invention provides a gas sensor that accurately and rapidly detects the presence of low concentrations of ethanol vapor in a cabin of a motor vehicle employing a source of infrared radiation, a first detector configured to receive infrared radiation from the source in a first region of the electromagnetic spectrum and a second detector for detecting a parameter, such as an amount of radiation received from the source in a second region of the electromagnetic spectrum, a temperature of the source and/or an amount of a second gas present in the cabin, affecting the amount of infrared radiation detected by the first detector. With this data, the presence of ethanol vapor in a cabin is established by an output of the gas sensor based on signals from both the first and second detectors. In any case, although described with reference to preferred embodiments, it should be readily understood that various changes or modifications could be made to the invention without departing from the spirit thereof. For example, features of one embodiment can be applied to the other embodiments. Additionally, gases other than ethanol vapor can be detected.

1. A gas sensor for sensing a presence of ethanol vapor in a cabin comprising:
a source of infrared radiation;
a first detector configured to detect an amount of ethanol vapor present in the cabin by detecting an amount of infrared radiation received from the source in a first region of the electromagnetic spectrum; and
a second detector configured to detect a parameter affecting the amount of infrared radiation detected by the first detector, wherein an output of the gas sensor is based on signals from both the first and second detectors.

2. The gas sensor of claim 1, wherein the parameter is established to correct for changes in the amount of infrared radiation received by the first detector.

3. The gas sensor of claim 1, wherein the parameter is an amount of radiation received from the source in a second region of the electromagnetic spectrum.

4. The gas sensor of claim 3, further comprising, in combination, a controller configured to correct for changes in infrared radiation received by the first detector due to changes in a temperature of the source by using the amount of infrared radiation received in the first region and the amount of infrared radiation received in the second region.

5. The gas sensor of claim 1, wherein the second detector constitutes a temperature sensor and the parameter is a temperature of the source of infrared radiation.

6. The gas sensor of claim 5, wherein the temperature sensor is coupled to the source of infrared radiation.

7. The gas sensor of claim 1, wherein the parameter is an amount of a second gas present in the cabin.

8. The gas sensor of claim 7, further comprising, in combination, a controller configured to correct for changes in infrared radiation received by the first detector due to changes in a temperature of the source by using the amount of the second gas present in the cabin.

9. The gas sensor of claim 8, wherein the controller is configured to correct for changes in infrared radiation received by the first detector due to changes in the temperature of the source by using:
a linear relationship of a ratio of the second gas to ethanol vapor as a function of temperature; or
a linear relationship of a ratio of ethanol vapor to the second gas as a function of temperature.

10. The gas sensor of claim 1, further comprising a first mirror and a second mirror, wherein the gas sensor is configured such that infrared radiation emitted by the source is reflected from the first mirror to the second mirror and from the second mirror to the first and second detectors.

11. The gas sensor of claim 10, further comprising:
a filter configured to reflect infrared radiation from the first detector to the second detector; or
a splitter configured to send infrared radiation, reflected from the second mirror, to the first and second detectors.

12. The gas sensor of claim 1, further comprising: a first tube, a second tube and a mirror, wherein:
the source is located at a first end of the first tube;
the first detector is located at a first end of the second tube;
the mirror is located at a second end of one of the first and second tubes; and
infrared radiation emitted by the source travels in a first direction from the source to the mirror and is reflected in a second direction, different from the first direction, to the first detector.

13. The gas sensor of claim 1, further comprising a taper coupled to the first detector, wherein the taper is configured to reject unwanted reflections of infrared radiation.

14. The gas sensor of claim 1, wherein the gas sensor is configured to detect the amount of ethanol vapor present in the cabin without using a concentrator configured to collect ethanol vapor into a concentrated form.

15. A method of sensing a presence of ethanol vapor in a cabin comprising:
emitting infrared radiation with a source;
detecting, with a first detector, an amount of infrared radiation received from the source in a first region of the electromagnetic spectrum; and
detecting, with a second detector, a parameter affecting the amount of infrared radiation detected by the first detector; and
determining an amount of ethanol vapor present in the cabin based on signals from both the first and second detectors.

16. The method of claim 15, further comprising: utilizing the parameter to correct for changes in the amount of infrared radiation received by the first detector.

17. The method of claim 15, wherein the parameter is an amount of radiation received from the source in a second region of the electromagnetic spectrum.

18. The method of claim 17, further comprising: correcting for changes in infrared radiation received by the first detector due to changes in a temperature of the source by using the amount of infrared radiation received in the first region and the amount of infrared radiation received in the second region.

19. The method of claim 15, wherein the second detector constitutes a temperature sensor and the parameter is a temperature of the source of the infrared radiation.

20. The method of claim 15, wherein the parameter is an amount of a second gas present in the cabin.

21. The method of claim 20, further comprising: correcting for changes in infrared radiation received by the first detector due to changes in a temperature of the source based on the amount of the second gas present in the cabin.

22. The method of claim 21, further comprising, when correcting for changes in infrared radiation received by the first detector due to changes in the temperature of the source, using:
a linear relationship of a ratio of the second gas to ethanol vapor as a function of temperature; or
a linear relationship of a ratio of ethanol vapor to the second gas as a function of temperature.

23. The method of claim 15, further comprising:
reflecting infrared radiation emitted by the source from a first mirror to a second mirror; and
reflecting infrared radiation from the second mirror to the first and second detectors.

24. The method of claim 23, further comprising:
reflecting infrared radiation from the first detector to the second detector; or
sending infrared radiation, reflected from the second mirror, to the first and second detectors using a splitter.

25. The method of claim 15, wherein the source is located at a first end of a first tube, the first detector is located at a first end of a second tube and a mirror is located at a second end of one of the first and second tubes, and wherein emitting infrared radiation with the source includes emitting infrared radiation in a first direction, the method further comprising:
reflecting the infrared radiation emitted by the source with the mirror such that the infrared radiation travels in a second direction, different from the first direction, to the first detector.

26. The method of claim 15, further comprising:
rejecting unwanted reflections of infrared radiation with a taper coupled to the first detector.

27. The method of claim 15, wherein the amount of ethanol vapor present in the cabin is detected without using a concentrator configured to collect ethanol vapor into a concentrated form.
